(19) United States
(12) Patent Application Publication
(10) Pub. No.: US 2018/0148754 A1
(43) Pub. Date: May 31, 2018

(54) OPTIMISED METHOD FOR DECONTAMINATING THE STARCH USED AS A RAW MATERIAL FOR OBTAINING GLUCOSE POLYMERS INTENDED FOR PERITONEAL DIALYSIS

(71) Applicant: Roquette Freres, Lestrem (FR)

(72) Inventors: Pierre Lanos, La Bassee (FR); Thierry Dupont, Vendin Les Bethune (FR); Fabrice Allain, Lille (FR); Mathieu Carpentier, Saint Andre Lez Lille (FR); Héla Hacine-Gherbi, Villeneuve-d'Ascq (FR); Agnès Denys, Lille (FR)

(73) Assignee: Roquette Freres, Lestrem (FR)

(21) Appl. No.: 15/578,152

(22) PCT Filed: Jun. 3, 2016
(86) PCT No.: PCT/FR2016/051325

§ 371 Date: Nov. 29, 2017

(30) Foreign Application Priority Data
Jun. 4, 2015 (FR) 1555077

(51) Int. Cl. C12P 19/04; C12P 19/14; A61K 31/718; C08B 30/04

(52) U.S. Cl. CPC C12P 19/04; C08B 30/044; A61K 31/718; C12P 19/14

(57) ABSTRACT

The present invention concerns a method for decontaminating the starches used as a raw material for the preparation of glucose polymers intended for peritoneal dialysis, the method comprising the following steps: —preparing a Waxy corn starch, —placing the Waxy starch in suspension at a concentration of between 20 and 40% dry matter in a process water at a pH of between approximately 5 and approximately 6, in particular approximately 5.5, —treating the starch suspension with a peracetic acid solution at a concentration equal to or between 100 and 500 ppm, preferably 300 ppm, —dewatering the starch, then dissolving in demineralised water adjusted to a pH of between approximately 5 and approximately 6, in particular approximately 5.5 and at a concentration of between 20 and 40% dry matter, —increasing the temperature to 107° C., then adding an alpha-amylase for 15 minutes, —optionally, treating with an enzymatic preparation having detergent and clarification properties, —filtering the suspension on a bed of diatoms, —treating with an active carbon having a very high adsorption capacity, of pharmaceutical quality, and of “microporous” Porosity, —treating with a second active carbon of “mesoporous” porosity, —optionally, passing over a macroporous adsorbent polymer resin, having a porosity greater than 100 angstroms, —optionally, continuous 5000 Da ultrafiltration, —safety filtration through a sterile filter having a porosity of 0.22 μm.

The present invention relates to the development of an optimized method for decontaminating starches used in circuits for producing glucose polymers, more particularly those intended for the medical fields, more particularly still to that of peritoneal dialysis.

### TECHNICAL BACKGROUND OF THE INVENTION

The Applicant company has chosen to develop its invention in a field which is known for the dangerousness of the contaminants of microbial origin capable of developing in the glucose polymer production circuits and which are the source of possible inflammatory reactions which are very harmful to human health.

In the context of a health safety approach, it is important to ensure the absence of contaminants of microbial origin, both in the form of living cells and in the form of cell debris, by all appropriate technical means, especially:
the definition of methods for effective identification and assaying of contaminants,
the definition of safe production circuits, by setting up appropriate purification devices and techniques.

In the case of peritoneal dialysis, a certain number of ingredients must be prepared under the strictest conditions of purity.

This is because peritoneal dialysis is a type of dialysis, the aim of which is to remove waste such as urea, creatinine, excess potassium or surplus water that the kidneys cannot manage or can no longer manage to purify out of the blood plasma. This medical treatment is indicated in the event of end-stage chronic renal failure.

The dialyzates most commonly used are composed of a buffer solution (lactate or bicarbonate) at acidic pH (5.2-5.5) or physiological pH (7.4) to which are added:
electrolytes (sodium, calcium, magnesium, chlorine) and most importantly
an osmotic agent, principally a glucose polymer, such as for example “icodextrin” present in the ambulatory peritoneal dialysis solution EXTRANEAL® sold by BAXTER.

In this more particular field of the use of glucose polymers intended for continuous ambulatory peritoneal dialysis, it very quickly became apparent that these starch hydrolyzates (mixture of glucose, and of glucose oligomers and polymers) could not be used as is.

European patent application EP 207 676 teaches that glucose polymers forming clear and colorless solutions at 10% in water, having a weight-average molecular weight (Mw) of 5 000 to 100 000 daltons and a number-average molecular weight (Mn) of less than 8 000 daltons, are preferred.

Such glucose polymers also preferably comprise at least 80% of glucose polymers of which the molecular weight is between 5 000 and 50 000 daltons, little or no glucose or glucose polymers with a DP less than or equal to 3 (molecular weight 504) and little or no glucose polymers with a molecular weight greater than 100 000 (DP of about 600).

In other words, the preferred glucose polymers are glucose polymers with a low polydispersity index (value obtained by calculating the Mw/Mn ratio).

The methods proposed in patent application EP 207 676 for obtaining these glucose polymers with a low polydispersity index from starch hydrolyzates consist:
either in carrying out a fractional precipitation of a maltodextrin by means of a water-miscible solvent,
or in carrying out a molecular filtration of this same maltodextrin through various membranes possessing an appropriate cut-off or exclusion threshold.

In the two cases, these methods are aimed at removing both the very high molecular weight polymers and the low molecular weight monomers or oligomers.

However, these methods are not satisfactory both from the point of view of their implementation and from the point of view of the yields and the quality of the products that they make it possible to obtain.

In the interests of developing a method for producing a completely water-soluble glucose polymer with a low polydispersity index preferentially less than 2.5, preferably having an Mn of less than 8 000 daltons and having an Mw of between 12 000 and 20 000 daltons, which method lacks the drawbacks of the prior art, the Applicant company endeavored to solve this problem in its patent EP 667 356, by starting from a hydrolyzed starch rather than from a maltodextrin.

The glucose polymer obtained by chromatographic fractionation then preferably contains less than 3% of glucose and of glucose polymers having a DP less than or equal to 3 and less than 0.5% of glucose polymers having a DP greater than 600.

Risks of Contamination

However, it should be noted that there are risks of microbial contamination of the preparations intended for peritoneal dialysis.

Indeed, it is known that glucose polymer production circuits can be contaminated with microorganisms, or with pro-inflammatory substances contained in said microorganisms.

In the case in which the method for manufacturing glucose polymers starts from starch, it is conventionally described that, in starch production, the contamination of corn (or wheat) starches is due to microorganisms of the yeast, mold and bacteria type, and more particularly by acidothermophilic bacteria of the Alicyclobacillus acidocaldarius type (extremophilic bacteria which develop in the hot and acidic regions of the circuit).

The major risk for the patient who receives these contaminated glucose polymers is then peritonitis.

These episodes of peritonitis are caused by intraperitoneal bacterial infections, and the diagnosis is usually easily established through positive dialyzate cultures.

“Sterile peritonitis”, which is described as aseptic, chemical or culture-negative peritonitis, is, for its part, typically caused by a chemical irritant or a foreign body.

Since the introduction of icodextrin for the preparation of peritoneal dialysis solutions, isolated cases of aseptic peritonitis have been reported that may be linked to various causes, especially induction by pro-inflammatory substances potentially present.

Aseptic inflammatory episodes are therefore major complications observed after injections of dialysis solutions.

While some of these inflammatory episodes are linked to a problem of chemical nature (accidental injection of chemical contaminants or incorrect doses of certain compounds), the majority of cases are directly associated with the presence of contaminants of microbial origin that are present in the solutions used to prepare the dialysis solutions.

Lipopolysaccharides (LPSs) and peptidoglycans (PGNs) are the main contaminants of microbial origin with a high risk of triggering an inflammation, even when they are present in trace amounts.

It is, moreover, to the Applicant company's credit to have also taken into account the presence of molecules capable of exacerbating the inflammatory response induced by these contaminants, such as PGN depolymerization products, the minimum structure of which that is still bioactive being muramyl dipeptide (MDP).

In addition to the PGN depolymerization products, formylated microbial peptides, the prototype of which is f-MLP (formyl-Met-Leu-Phe tripeptide), also have a substantial synergistic activity. Originally, these peptides were identified for their chemoattractant activity on leukocytes, although they are incapable of inducing a cytokine response per se.

It is therefore important not to overlook these “small molecules”, since they can indirectly account for aseptic inflammatory episodes by exacerbating the effects of traces of PGN and/or of LPS.

Definition of Methods for Effective Identification and Assaying of Said Contaminants

The Applicant company has therefore applied itself to developing detection and assaying methods which are more effective than those accessible in the prior art.

Over the last few years, many tests using primary cells have been developed in order to replace animal models in inflammatory response tests.

However, these in vitro models are subject to considerable interindividual variability, which can be responsible for experimental biases.

Conversely, monocyte cell lines give consistent responses, thereby explaining why the tests currently being developed increasingly use cells of this type in culture. However, these tests have the drawback of giving an overall inflammatory response to all the contaminants present as a mixture in a solution, and consequently do not make it possible to characterize the nature of the contaminant.

It is also important to note that the exacerbated inflammatory response is visible for cytokines of the acute phase of the inflammation, such as:
TNF-α (Tumor Necrosis Factor alpha),
IL-1β (interleukin 1β) and
chemokines such as CCL5 (Chemokine (C-C motif) ligand 5)/RANTES (Regulated upon Activation, Normal T-cell Expressed and Secreted),

but is not, or barely, visible for IL-6 (interleukin 6).

Thus, methods based on the production of IL-6 (US 2009/0239819 and US 2007/0184496) are not suitable for detecting contaminants as a mixture in a solution.

It was therefore to the Applicant company's credit to have developed, in its international patent application WO 2012/143647, sensitive and effective methods for detecting microbial contaminants which have a pro-inflammatory action, below the threshold of sensitivity of the procedures currently used and/or described in the literature, and subsequently to have identified the family, or even the nature, of the pro-inflammatory molecules present in trace amounts in the batches originating from the production circuits.

Determining the Effectiveness of the Individual Purification Steps

The Applicant company then sought to better define the key purification steps to be carried out on the glucose polymers intended for peritoneal dialysis.

To this end, it applied itself to validating the key individual purification steps, by using the detection and assaying methods based on monocyte lines as presented in its international patent application WO 2012/143647.

Thus, in its international patent application WO 2013/178931, the Applicant company analyzed the effectiveness of the following individual steps carried out on glucose polymers intended for peritoneal dialysis:
heat treatment,
acidification,
passing over activated carbon,
passing over adsorption, ultrafiltration or filtration resins,
chemical or enzymatic hydrolysis.

In an as yet unexamined recent patent application, the Applicant company then endeavored to define a combination of several decontamination steps which were carefully selected and ordered for their effectiveness in eliminating all the inflammatory molecules likely to be present in the glucose polymers resulting from the manufacturing method, regardless of the nature of the contamination. The method of this invention thus relates to the following combination of steps, carried out on glucose polymers intended for peritoneal dialysis:
treating with an enzymatic preparation with detergent and clarifying properties, treating with a pharmaceutical-grade activated carbon with very high adsorption capacity and “microporous” porosity;
optionally treating with a second activated carbon with “mesoporous” porosity,
passing over a macroporous adsorbent polymer resin having porosity of greater than 100 angstrom, and
continuous 5 kDa ultrafiltration.

While this work has made it possible to define the best combination able to make glucose polymers for peritoneal dialysis safe from all potential contaminants, there is still an unmet need for the upstream development of an optimized method for decontaminating the actual source of potential contaminants of the circuits for producing glucose polymers for peritoneal dialysis, namely waxy corn starch and crude starch hydrolyzate, which enter into the circuit for the preparation of said glucose polymers.

Indeed, the provision of a method able to effectively treat waxy corn starch naturally contaminated by cell debris from microorganisms of the yeast, mold or bacteria type would then make it possible to simplify any subsequent treatment of the glucose polymers which result therefrom.

Such a decontamination treatment at the source on crude products will effectively lead to reducing the load of contaminants likely to pollute the glucose polymer production circuits and will thereby contribute to making them safe, making it possible to satisfy the prerequisites of the pharmaceutical industry in terms of the degree of purification of products intended for peritoneal dialysis.

### DETAILED DESCRIPTION OF THE INVENTION

The present invention therefore provides a combination of several carefully selected and ordered decontamination steps which prove effective in eliminating all the inflammatory molecules likely to be present in the starches and starch hydrolyzates used as raw material for the preparation of glucose polymers intended for peritoneal dialysis, regardless of the nature of the contamination.

The method according to the invention for the decontamination of starches used as raw material for the preparation of glucose polymers intended for peritoneal dialysis, the method comprising the following steps:
preparing a waxy corn starch,
suspending the waxy starch at a concentration of between 20 and 40% dry matter in a process water at a pH of between approximately 5 and approximately 6, in particular approximately 5.5, treating the suspension of starch with a solution of peracetic acid at a concentration of between 100 and 500 ppm, preferably of 300 ppm,
removing excess water from the starch then taking up in a demineralized water adjusted to a pH of between approximately 5 and approximately 6, in particular approximately 5.5 and at a concentration of between 20 and 40% of dry matter,
raising the temperature between approximately 100° C. and 110° C., preferably to approximately 107° C., then adding α-amylase for approximately 10 to 20 minutes, preferably approximately 15 minutes,
optionally treating with an enzymatic preparation with detergent and clarifying properties,
filtering the suspension over a bed of diatoms,
treating with a pharmaceutical-grade activated carbon with very high adsorption capacity and “microporous” porosity, treating with a second activated carbon with “mesoporous” porosity,
optionally passing over a macroporous adsorbent polymer resin having a porosity of greater than 100 angstrom, and
optionally, continuous 5 kDa ultrafiltration,
safety filtration over a sterile filter with a porosity of 0.22 μm.

The steps of the method are to be carried out in the order in which they appear.

In a preferred embodiment, all the steps of the method, including the optional steps, are carried out.

Within the context of the invention:
“process water” means all or part of the water used in the wet starch production circuit which is recycled therein (cf. especially diagram 5 of the document Bilan énérgétique des industries de transformation des céréales dans la CEE [Energy balance of the cereal processing industries in the EEC], available on the Internet at the file address ///J:/CDNA10994FRC_001.pdf)
“enzymatic preparation with detergent and clarifying properties” means enzymatic activity of mannanase type, such as Mannaway® sold by Novozymes;
“pharmaceutical-grade activated carbon with very high adsorption capacity and ‘microporous’ porosity” means an activated carbon with porosity equivalent to Norit C Extra USP activated carbon;
“activated carbon with ‘mesoporous’ porosity” means an activated carbon with porosity equivalent to ENO-PC activated carbon;
“macroporous adsorbent polymer resin having porosity of greater than 100 angstrom” means a resin of DOWEX OPTIDORE SD2 type.
“approximately” means plus or minus 10% of the value, preferably plus or minus 5%. For example, approximately 100 means between 90 and 110, preferably between 95 and 105. This also refers to the exact value.

The pro-inflammatory contaminants are above all molecules of bacterial origin.

They may be, in particular:
PGNs,
LPSs,
lipopeptides,
PGN depolymerization products, especially MDP,
formylated microbial peptides, such as f-MLP,
β-glucans,
etc.

The methods for measuring the in vitro inflammatory responses which are used in the context of the present invention to monitor the effectiveness of the decontamination steps of the methods for preparing glucose polymers for therapeutic use in humans (e.g. peritoneal dialysis solutions) are based on cell tests (“bio-assays”) using lines of monocyte/macrophage type (THP-1, and/or Raw-BlueT^{M}) and transfected lines expressing a specific natural immunity receptor (HEK-Blue™), which cell tests were developed by the Applicant company from commercial cell lines and detailed in its prior patent applications.

Five lines are thus preferably used:
Raw-Blue™ line: this line, derived from mouse macrophages, responds to the majority of the pro-inflammatory contaminants that may be present in the glucose polymer matrices and derivatives (PGN, lipopeptides, LPS, zymosan, LTA). Its use therefore makes it possible to estimate the overall load of pro-inflammatory molecules present in the samples.
HEK-Blue™ hTLR2 line: this line, expressing the hTLR2 receptor, specifically responds to TLR2 agonists (PGN and lipopeptides especially). Its use therefore makes it possible to determine the level of these contaminants in the triggering of inflammatory responses.
HEK-Blue™ hTLR4 line: this line, expressing the hTLR4 receptor, specifically responds to LPSs. Its use therefore makes it possible to determine the level of these contaminants in the triggering of inflammatory responses.
HEK-Blue™ hNOD2 line: this line, expressing the hNOD2 receptor, specifically responds to NOD2 agonists. Its use therefore makes it possible to determine the level of MDP and related molecules in the triggering of inflammatory responses.
HEK-Blue™ Null2 line: this is a control line which has not been transfected with an immunity receptor. Its use is necessary to verify that the solutions of glucose polymers or of hydrolyzates thereof do not induce SEAP production via a toxicity mechanism.

However, it should be noted that those skilled in the art may also use other commercial lines (IMGENEX) or they may prepare them.

In one preferred embodiment, the cell lines are used at a density between 0.5 and 1×10⁶ cells/ml of culture medium, and the bringing of the preparation of glucose polymers or hydrolyzates thereof into contact with the cells lasts approximately 16 to 24 h.

The contaminants may be quantified using a dose-response curve. This dose-response curve may especially be produced with the same cells, under the same conditions, with increasing doses of contaminants. The dose-response curves are in particular produced with LPS, PGN, lipopeptide and MDP standards.

Preferably, such a dose-response curve can be produced for cells expressing TLR4 (for example, THP-1, HEK-Blue™ hTLR4 and Raw-Blue™) with increasing doses of LPS, for cells expressing TLR2 (for example, THP-1, HEK-Blue™ hTLR2 and Raw-Blue™) with increasing doses of PGN, and for cells that are reactive via NOD2 (for example, HEK-Blue™ hNOD2) with increasing doses of MDP.

The cell tests may be carried out as described in the Applicant's patent applications: WO2012/143647 and WO2013/178931.

After the preparation of the suspension of waxy starch at a concentration of between 20 and 40% dry matter in a process water at a pH of between approximately 5 and approximately 6, in particular approximately 5.5, the first actual decontamination step of the method in accordance with the invention consists in treating with peracetic acid. The peracetic acid can be used at a concentration of between 100 and 500 ppm. Preferentially, a water treated with 300 ppm of peracetic acid will be taken. The contact time is approximately 2 hours at a temperature between approximately 5 and approximately 15° C., preferably approximately 10° C. This treatment of starch with peracetic acid has demonstrated effectiveness in reducing contaminants at any subsequent step of the method, especially with respect to PGN-type contaminants. This effect is all the more marked when process water is used to prepare the suspension of starch.

The following step, after removing excess water from the starch and then taking up the latter in a demineralized water adjusted to a pH of between approximately 5 and approximately 6, in particular approximately 5.5 and at a concentration of between 20 and 40% of dry matter, is the liquefaction of the starch. The starch is liquefied by placing the suspension at a temperature of between approximately 100° C. and 110° C., preferably at approximately 107° C., and adding α-amylase. The enzymatic hydrolysis lasts for approximately 10 to 20 minutes, preferably approximately 15 minutes.

The following step may optionally consist in treating with an enzymatic preparation with detergent and clarifying properties.

This enzymatic activity of mannanase type, such as the enzymatic preparation Mannaway® sold by Novozymes, which has proven effective for dissociating macrocomplexes such as bacterial debris and high molecular weight PGNs.

It will therefore be implemented if the analyses of the liquefied and hydrolyzed starch reveal a high level of PGN-type contaminants.

The activity of this enzymatic preparation is optimal when it is used at a final concentration of 0.4% (vol/vol) in the 32% (weight/vol) glucose polymer solution, adjusted to pH 10 with NaOH, for a treatment time of 24 h at 50° C.

After treatment, the solution is filtered over a bed of diatoms, as will be exemplified below.

The following step then consists in treating with two activated carbons in cascade:

1) a first pharmaceutical-grade activated carbon with very high adsorption capacity and “microporous” porosity.
The Applicant company recommends using an activated carbon of Norit C Extra USP type. This is because C Extra USP carbon proves effective in eliminating PGNs and their degradation products.
Its action is optimal when it is added at the final concentration of 0.5% (weight/volume) in the 32% (weight/vol) glucose polymer solution, adjusted to pH 4.5 with HCl.
The treatment is carried out with stirring for 1 h at 80° C.
2) a second activated carbon with “mesoporous” porosity.
Here, an activated carbon of ENO-PC type is preferred. This quality of activated carbon has a broad spectrum of action and makes it possible preferentially to eliminate molecules with a molecular weight of <100 kDa (for example, LPSs and degradation products of PGNs).

It is also used here at a content of 0.5% at pH 4.5 for 1 h at a temperature of 80° C.

The solution obtained after passing over these two activated carbons is finally filtered over a membrane with a porosity threshold of 3 μm.

The optional following step consists in treating over a macroporous adsorbent polymer resin having a porosity of greater than 100 angstrom.

Dowex SD2 resin is chosen, which has a broader spectrum of elimination of contaminating molecules (other than PGNs) than other resins of the same family.

As will be exemplified below, 32% glucose polymer solutions (250 ml) are eluted on a column containing 20 ml of this resin.

This step is recommended for raw materials heavily loaded with LPSs and/or treated with an enzymatic preparation with detergent and clarifying properties.

Also optionally, the following step consists of continuous ultrafiltration on a membrane having a cut-off threshold at 5 kDa.

This step is recommended for raw materials heavily loaded with PGN depolymerization products.

The final step consists of a safety filtration over a membrane having a cut-off threshold of 0.22 μm.

The invention will be understood more clearly from the following examples which are intended to be illustrative and nonlimiting.

### EXAMPLES

### Example 1: Characteristics of Cell Lines Used for the Inflammatory Response Tests

The dose-response curves are produced with standard agonist molecules: LPS, PGN and MDP, dissolved in a solution of uncontaminated maltodextrin (PGN <1 ng/g, LPS <0.5 ng/g, MDP <0.2 ng/g) at 32% (weight/volume) in apyrogenic water (for injection), according to the teaching of the international patent application WO 2013/178931 from the Applicant company.

The Raw-Blue™ and HEK-Blue™ hTLR2, hTLR4, hNOD2 and Null2 cells are incubated with increasing concentrations of agonists, and the cell response is measured by quantifying the SEAP activity:
RawBlue™ line: the cells respond to the major inflammatory molecules liable to be present in the glucose polymer matrices and derivatives (PGN and LPS); they especially have high reactivity with respect to PGNs, but do not respond to its depolymerization products (MDP).
HEK-Blue™ hTLR2 line: high reactivity with respect to PGNs; the cells show no reactivity with respect to LPSs and MDP,
HEK-Blue™ hTLR4 line: high reactivity with respect to LPSs; the cells show no reactivity with respect to PGNs and MDP,
HEK-Blue™ hNOD2 line: high reactivity with respect to MDP; the cells show no reactivity with respect to PGNs and LPS,
HEK-Blue™ Null2 line: control for absence of cellular toxicity; the cells show no reactivity with respect to PGNs, LPSs and MDP.

### Example 2: Preparation of the Glucose Polymer Raw Materials

The raw material preparation steps were all carried out on the pilot scale.

The raw materials are prepared from waxy starch suspended at a concentration of between 20 and 40% dry matter (weight/volume).

The starch in suspension is left overnight at 4° C., excess water is removed therefrom, then it is resuspended in water adjusted to pH 5.5 at a concentration of between 20 and 40%. The suspension is then heated to 107° C., then treated in the presence of α-amylase for 15 min. After liquefaction, the enzymatic reaction is stopped by addition of 1N HCl (pH 4), and the liquefaction products are filtered on a bed of diatoms (40 μm).

Depending on the tests, the starch is dissolved in demineralized water or process water, so as to estimate the proportion of contamination contributed to the raw materials by this commonly used water.

In order to reduce the load of contaminants at the beginning of the method, the starch suspension can be treated with a 0.03% peracetic acid solution. In this case, the waxy starch is suspended at a concentration of between 20 and 40% (w/v), left overnight at 4° C., excess water is removed therefrom, it is resuspended and then treated in the presence of peracetic acid (300 ppm). After another removal of excess water, the starch is resuspended in demineralized water adjusted to pH 5.5 at a concentration of between 20 and 40% (w/v). As before, the solution is heated, α-amylase is added for 15 min. The reaction is stopped by adding 1N HCl (pH 4) and then filtered.

The teachings of the international patent application WO 2013/178931 from the Applicant company have shown that the enzymatic preparation Mannaway® is effective in dissociating macrocomplexes such as bacterial debris and high molecular weight PGNs in a final glucose polymer preparation. Its activity is optimal when it is used at a final concentration of 0.4% (vol/vol) in a 32% (weight/vol) glucose polymer solution adjusted to pH 8 with NaOH, for a treatment time of 24 hours at 50° C. After treatment, the solution is neutralized with HCl and the enzyme is inactivated by heating at 85° C. for 10 min. However, the Mannaway® enzymatic preparation is contaminated with traces of LPS. In addition, traces of enzyme may remain after treatment. In order to take these exogenous contaminations into account, the enzymatic treatment step is placed at the end of the preparation of the raw materials before the filtration and consequently before the start of the decontamination procedure.

After each step, samples are taken to analyze the overall inflammatory load (test with Raw-Blue™ cells) and the amounts of PGN, LPS and MDP contaminants (HEK-Blue™ cell responses).

### Example 3: Comparison of Inflammatory Responses Induced by the Raw Materials Before Decontamination

The aim of these tests is to determine the pro-inflammatory reactivity of the raw materials, to identify the nature of the biocontaminants, and to test the means making it possible to reduce their before carrying out the decontamination procedure. The presence of biocontaminants in the various raw materials is analyzed by means of the five cell types, so as to have an overview of the inflammatory responses specific to certain contaminants:
Raw-Blue™ line: any contaminants with high reactivity for PGNs,
HEK-Blue™ hTLR2 line: high reactivity for PGNs and lipopeptides,
HEK-Blue™ hTLR4 line: high reactivity for LPSs,
HEK-Blue™ hNOD2 line: MDP and PGN depolymerization products,
HEK-Blue™ Null2 line: control for absence of cell toxicity.

For these cell tests, the raw materials are diluted in the culture medium of the cells to obtain a final concentration equal to 3.2% (w/v). The results are expressed as activity (SEAP response) relative to the maximum cell response.

Test 1:

The waxy starch was taken up at 20% in water at pH 5.5, then treated in the presence of α-amylase. Two preparations are tested:
preparation 1: waxy starch+demineralized water (WD)
preparation 2: waxy starch+process water (WR)

Samples were taken after each stage of the method: suspension of the starch in water, addition of α-amylase (E), liquefaction (DE). The results of the cell tests are given in FIG. 1.

The suspension of the starch in demineralized water (WD) released small amounts of PGN and LPS into the supernatant (moderate responses in HEK-TLR2, HEK-TLR4 and Raw cells). The addition of the enzyme (WD+E) did not result in contamination. On the other hand, liquefaction released large amounts of contaminants, as evidenced by the strong responses of the DEWD sample, measured with HEK-TLR2 and HEK-TLR4. These results indicate that PGNs and LPSs are associated with the starch grains and that the liquefaction has caused their release.

In comparison with demineralized water, process water contains high amounts of PGN, since the TLR2 responses are saturated. A smaller increase for the response of HEK-TRL4 cells was observed, indicating the presence of LPS in this process water, but at lower levels than the PGNs.

The responses of the HEK-NOD2 cells are not significant for both preparations, or relatively low for the response observed after liquefaction of the starch taken up in the process water. This observation suggests that PGNs are not particularly degraded, and are therefore essentially present in the form of large complexes.

To verify this hypothesis, the samples taken were filtered on microfiltration units of the Centricon 30 type (cut-off threshold 30 kDa). Cell tests with HEK-TLR2 and HEK-TLR4 were carried out on the filtrates and the responses were compared with those obtained with the unfiltered products. The results are shown in FIG. 2.

The sample originating from the suspension of the starch in process water (WR) contains high amounts of PGN and LPS, which are not found in the corresponding filtrates. The process water alone (water R) also induced strong responses in the HEK-TLR2 and HEK-TLR4 cells, proof that the contaminants of LPS and PGN type present in the WR sample taken predominantly originate from the suspension step. Furthermore, the moderate responses observed with the suspension in demineralized water (WD) confirm that the non-liquefied starch releases few contaminants.

Conversely, the two samples originating from the liquefaction of the starch (DEWD and DEWR) are highly contaminated. No significant trace of PGN or LPS is found in the filtrates, which confirms their presence as molecules or aggregates with a molecular weight >30 kDa. These data are therefore in support of the presence of large molecules, aggregates and/or cell debris, contributed by the process water and/or released from the starch grains by the liquefaction step.

Test 2:

In this test, the effect of peracetic acid was evaluated by carrying out the experiments on the same batch of starch dissolved in demineralized water.

The waxy starch was taken up at 20% in water at pH 5.5, then treated in the presence of α-amylase. Two preparations were tested:
preparation 1: waxy starch+demineralized water (WD)
preparation 2: waxy starch+demineralized water followed by treatment with peracetic acid (WAD).

Samples were taken after each stage of the method: suspension of the starch in water, addition of α-amylase (E), liquefaction (DE). The results of the cell tests are given in FIG. 3.

The suspension of the starch in demineralized water (preparation 1) released small amounts of PGN and LPS into the supernatant (responses from the WD sample with HEK-TLR2, HEK-TLR4 and Raw). The addition of the enzyme did not cause any contamination. On the other hand, liquefaction released large amounts of biocontaminants, as evidenced by the strong responses obtained with the DEWD sample in the HEK-TLR2 and HEK-TLR4 tests. These results confirm that PGN and LPS are strongly associated with starch grains and that liquefaction causes them to dissolve.

The results obtained with the preparation 2 show that the peracetic acid had a neutralizing effect on the PGNs associated with the starch grains. Indeed, there is no TLR2 response in the samples before liquefaction since the values obtained with the WAD and WAD+E samples are at the detection threshold of the assay. In addition, the TLR2 response is significantly reduced after liquefaction (DEWAD versus DEWD).

On the other hand, treatment has little effect on LPS since the TLR4 responses obtained with the preparation 2 are similar to those observed in the absence of peracetic acid for all the samples. This data indicates that LPSs are not very sensitive to treatment with peracetic acid. In addition, the presence of these contaminants explains why eliminating the PGNs induces only a moderate decrease in the inflammatory response observed with the Raw cells.

The HEK-NOD2 responses are not significant. This observation indicates that the action of peracetic acid is not accompanied by the formation of potentially inflammatory degradation products, such as small fragments of PGN and/or depolymerization products of MDP type, but indeed by neutralization of the inflammatory activity of the PGNs.

Test 3:

In this test, the effect of the peracetic acid was evaluated by carrying out the experiments on the same batch of starch dissolved in the process water.

Waxy starch was taken up to approximately 30% dry matter in water at pH 5.5, then treated in the presence of α-amylase on a jet cooker. Two preparations were tested:
preparation 1: waxy starch dissolved in process water, left overnight at 4° C., excess water is removed therefrom, then it is taken up in process water adjusted to pH 5.5 (WR).
preparation 2: waxy starch dissolved in process water, left overnight at 4° C., then treated with peracetic acid at 300 ppm. Removing excess water again, then taking up in demineralized water adjusted to pH 5.5 (WRAD).

Preparation 1 therefore corresponds to the standard protocol. In preparation 2, the starch is taken up in demineralized water after treatment with peracetic acid so as not to introduce new contaminants.

Samples were taken after the steps of suspending the starch in the process water and liquefaction. The results of the cell tests are given in FIG. 4.

As expected, the process water contributed a significant amount of soluble PGNs in preparation 1 (TLR2 response for the WR sample). After liquefaction, the TLR2 response is saturated (DEWR), which confirms the release of contaminants of PGN type associated with the starch grains. In comparison, the peracetic acid treatment was very effective in reducing the load of PGN in the preparation 2, whether contributed by the water (WRAD) or released by liquefaction (DEWRAD).

The process water also contributed a large amount of soluble LPSs (TLR4 response in the WR sample), but unlike that which is observed with the PGNs, liquefaction released less of this type of contaminant (TLR4 responses for WR and DEWR samples). In comparison, the peracetic acid had a moderate effect on the load of LPS, since a slight decrease in the contaminant load contributed by the process water is observed in the preparation 2 (WRAD).

In both preparations, the water is not loaded with MDP or with PGN fragments, and liquefaction released a small amount thereof (similar NOD2 responses for DEWR and DEWRAD).

Finally, the responses of the Raw cells reflecting the overall inflammatory load are reduced in the samples after peracetic acid action, which is in agreement with the significant loss of PGN in the preparation 2 (WRAD versus WR and DEWRAD versus DEWR). The residual reactivity of the Raw cells after action of the peracetic acid is therefore predominantly due to the LPSs contributed by the process water.

Overall, this data demonstrates the effectiveness of the treatment by peracetic acid in significantly reducing the load of PGN in the raw material before the decontamination procedure. The advantage of the decontamination continues in the subsequent steps of the method.

Test 4:

The first tests suggest that the majority of the inflammatory molecules contributed by the process water and/or released from the starch grains by the liquefaction step are present in the form of high molecular weight complexes such as aggregates and/or cell debris.

In this new test, a treatment with the Mannaway® enzyme was added between the liquefaction and filtration steps, due to the effectiveness of this enzymatic preparation in dissociating high molecular weight aggregates and PGNs.

Waxy starch was taken up at approximately 30% in process water at pH 5.5, left overnight, and then treated with peracetic acid at 300 ppm. After removing excess water then taking up in demineralized water adjusted to pH 5.5, α-amylase was added for the liquefaction step (DEWRAD). The solution was then adjusted to pH 8, then treated in the presence of the Mannaway® enzymatic preparation (0.4%) for 24 h at 50° C. Finally, the solution was filtered on a bed of diatoms (DEWRADM).

Samples were taken after the steps of treatment with peracetic acid (WRAD), liquefaction (DEWRAD) and action of the enzymatic preparation Mannaway® (DEWRADM). The results of the cell tests are given in FIG. 5.

As expected, the process water contributed a significant amount of soluble PGNs in the preparation (WRAD). In this test, water must have been particularly contaminated with PGN, since the TLR2 and Raw responses are still very high after peracetic acid (WRAD) action, and were largely saturated after liquefaction (DEWRAD). However, it is possible to note a decrease in the Raw response after action of Mannaway®, which is proof that the enzymatic preparation did indeed eliminate a portion of the inflammatory contaminants (DEWRADM versus DEWRAD).

Unlike PGN, the load of LPS is hardly modified after liquefaction, proof that the predominant portion is contributed by the process water. On the other hand, after addition of Mannaway®, there is a strong increase in TLR4 response, which was predictable given that this solution is itself contaminated with LPSs.

This last result indicates that this exogenous contribution of LPS will certainly have to be taken into account during the decontamination procedure.

### Example 4: Effect of Decontamination Procedures on Inflammatory Responses Induced by the Raw Materials

Various decontamination treatments of glucose polymers (in the form of a finished product) have already been tested individually and in combination, and reported in the international patent application WO 2013/178931 from the Applicant company. This work made it possible to identify the treatments best suited to each type of contaminants present in the samples and to determine the conditions for their application to glucose polymer matrices.

The inventors wanted to test the effectiveness of these treatments, developed on a finished product in the final purification step, on a complex mixture such as starch hydrolyzate.

The treatments selected are:
treatment on activated carbons: the activated carbons selected for the present study are: C extra USP, for its effectiveness in eliminating PGNs; ENO-PC, for its broad spectrum on contaminants of molecular weight <100 kDa (for example, LPS and PGN degradation products).

The action of the carbons is at its maximum when they are added at the final concentration of 0.5% (weight/volume) into the 32% (weight/volume) glucose polymer solution, adjusted to pH 4.5 with 1N HCl. The treatment is carried out with stirring for 1 h at 80° C. After treatment, the solution (500 ml) is neutralized by NaOH then filtered on a sintered glass filter (porosity of 3 μm). Given that the carbon treatments are carried out batchwise and require heating, neutralization and filtration steps, they are carried out before the other treatments.
passage over adsorption resins: The resins selected for the present study are: Dowex SD2, for its broad spectrum of contaminant elimination; MN-100, for its efficiency in retaining LPS-type molecules.

For the experiments, the 32% glucose polymer solutions (250 ml) are eluted on a column containing 20 ml of each resin. The teachings of the previous study have shown that this procedure does not cause any phenomenon of saturation of the resins by the glucose polymer solutions.
ultrafiltration on 5 kDa: the aim of the ultrafiltration treatment is to eliminate the small molecules (degradation products of PGNs and MDP) which are still present in the glucose polymer solutions. This step is therefore optional and used at the end of the procedure if the NOD2 response is positive.

The tests are carried out by continuously injecting the glucose polymer solution over a 5 kDa filter at a rate of 25 ml/min for 3 h at room temperature. To compensate for the loss of filtrate, the retentate is injected into the starting solution and continually adjusted to the initial volume (100 ml) by addition of sterile demineralized water.

The various decontamination steps were carried out in the laboratory. After each step, samples were taken under sterile conditions and used in the cell tests, so as to assay the overall inflammatory load (Raw response) and the amounts of biocontaminants (TLR2, TLR4 and NOD2 responses). For the saturated cell responses, the samples were diluted beforehand ( 1/10^{th} and 1/100^{th}).

Concentrations of contaminants were calculated by referring to the dose-response curves produced with standard agonist molecules: LPS, PGN and MDP, described in example 1 and established according to the teaching of the international patent application WO 2013/178931 from the Applicant company.

Contaminant concentrations were then reduced to the amount of glucose polymer present in the sample. Then, the values obtained after each decontamination step were compared with that of the starting raw material, so as to estimate the effectiveness of the decontamination procedures. The results are expressed as a percentage reduction relative to the initial load of contaminants and residual contamination relative to the threshold limits of detection (LOD) of each bio assay (in ng per g of glucose polymer): HEK-TLR2, <1 ng PGN; HEK-TLR4, <0.5 ng LPS; HEK-NOD2, <0.2 ng MDP; Raw, <2 ng PGN.

The following procedures were analyzed:

Procedure 1:

In this first decontamination test, the raw material was prepared following the protocol described in example 3, test 3: waxy starch (approximately 20%) dissolved in the process water, left overnight at 4° C. (WR), then treated with peracetic acid at 300 ppm. Removing excess water, then taking up in demineralized water at pH 5.5 (WRAD). Liquefaction, then filtering on a bed of diatoms (DEWRAD).

The raw material corresponding to the DEWRAD sample was then decontaminated using the following combination:

1. treatment with C extra USP carbon (0.5%), followed by filtration on a sintered glass filter (3 μm),

2. treatment with ENO-PC carbon (0.5%), followed by filtration on a sintered glass filter (3 μm),

3. passage on an SD2 resin column,

4. sterile filter filtration (0.22 μm).

Samples were taken after the various steps for preparing the raw material, then of the decontamination procedure.

The results of the cell tests are given in FIG. 6.

Firstly, treatment with peracetic acid (WRAD versus WR) reduced the PGN content before liquefaction (TLR2 and Raw responses) but did not have a significant effect on LPSs (TLR4 response). Liquefaction released large amounts of PGNs associated with the starch grains, since the TLR2 and Raw responses are saturated for the DEWRAD sample, while the TLR4 response is only very slightly increased. It can be concluded therefrom that in this test the starch was heavily loaded with PGN, since the TLR2 and Raw responses are saturated even after the action of peracetic acid (FIG. 6A).

To calculate the effectiveness of decontamination steps, the initial contaminant concentrations were determined from the DEWRAD sample for each cell type; for saturated cell responses (TLR2 and Raw), the sample was diluted beforehand to 11100^{t}h then the concentration values were corrected by the dilution factor. Residual concentrations of contaminants were calculated after each decontamination step, and the values were then related to the initial concentrations to express the results as a percentage reduction (FIG. 6B and table I).

Most surprisingly, the decontamination procedure enabled a very marked decrease in the TLR2 response, with a PGN load reduction of >99.9% (detection threshold). In addition, the C extra USP and ENO-PC carbons in series have an additive effect on the elimination of the PGNs before passing over resin, which reinforces the choice of these two carbons for their complementary action.

The procedure is also effective in reducing the NOD2 response, given that the threshold limit of detection is reached for this cell line at the end of the method. Compared with PGN, the reduction is only 90%, but this value is related to the fact that the NOD2 agonists (PGN and MDP depolymerization products) were present in trace amounts in the starting raw material.

The LPS elimination is >99.9% at the end of the procedure, and the TLR4 response also reaches the threshold limit of detection. It may be noted that the passage over the SD2 resin is to reach this decontamination threshold. Indeed, significant traces of LPS are still present after treatment with the two carbons. However, the material was highly contaminated with LPS, which may explain why the combined action of the two carbons was not sufficient to eliminate everything.

Finally, the response of the Raw cells confirms the effectiveness of this first procedure in eliminating all types of contaminants present in the raw material. Indeed, no significant inflammatory response (<threshold limit of detection) is observed any more, which reflects a >99.9% reduction in the overall inflammatory load.

TABLE I
Reduction values (as % of the initial load) and residual
load at the end of the decontamination method

| | HEK-TLR2 | HEK-TLR4 | HEK-NOD2 | Raw |
|---|---|---|---|---|
| Reduction (%) | >99.9 | >99.9 | >90 | >99.9 |
| Residual loads | <1 ng/g | <0.5 ng/g | <0.2 ng/g | <LOD |

LOD, limit of detection

Procedure 2:

the first test suggests that the SD2 resin can be used optionally with the proviso that the LPS content is not too high in the raw material. To test this hypothesis, a raw material was prepared following the protocol described above: waxy starch (approximately 20%) dissolved in the process water, left overnight at 4° C. (WR), then treated with peracetic acid at 300 ppm. Removing excess water, then taking up in demineralized water at pH 5.5 (WRAD). Liquefaction, then filtering on a bed of diatoms (DEWRAD).

The raw material corresponding to the DEWRAD sample was then decontaminated using a “simplified” combination without passage over SD2 resin.

1. treatment with C extra USP carbon (0.5%) followed by filtration on a sintered glass filter (3 μm),

2. treatment with ENO-PC carbon (0.5%) followed by filtration on a sintered glass filter (3 μm),

3. sterile filter filtration (0.22 μm).

Samples were taken after the various steps for preparing the raw material, then of the decontamination procedure.

The results of the cell tests are given in FIG. 7.

Before liquefaction, the TLR2 response obtained with the sample WR is saturated, indicating that the process water is highly contaminated with PGNs. The treatment with peracetic acid partially reduces this contamination (WRAD), but the liquefaction releases new PGNs, since the TLR2 response is once again saturated with the DEWRAD sample.

Conversely, LPS contaminations (TLR4 responses) remain moderate, whether they originate from the process water or from the liquefaction. The raw material used for this new procedure is therefore more heavily contaminated in PGN than the previous one, but less in LPS (FIG. 7A).

The DEWRAD sample was then treated according to the decontamination procedure combining the two carbons C-Extra and ENO-PC and the 0.22 μm filter filtration. The initial loads of contaminants contained in the DEWRAD sample were reduced to 100% and the relative reduction percentages were calculated from residual contaminant loads after each step (FIG. 7B).

The decontamination procedure enabled a very marked reduction in the TLR2 response (>99.9%), despite the high PGN contamination in the raw material and the lack of passage over SD2 resin. This result confirms the effectiveness of the carbons in eliminating this type of contaminant. The combination is also sufficient to reduce the load of PGN depolymerization products since the threshold limit of detection of the NOD2 response is reached at the end of the method.

Unlike the first decontamination test, the SD2 resin also does not appear to be necessary here to reduce LPS contamination. Indeed, the TLR4 response also reaches the threshold limit of detection, and the LPS elimination is >99.9% at the end of the procedure.

Finally, the response of the Raw cells confirms the effectiveness of this “simplified” procedure for eliminating the contaminants present in a raw material with a low LPS load. Indeed, there is no longer any significant inflammatory response at the end of the procedure (<threshold limit of detection).

Procedure 3:

In this third decontamination test, the raw material was prepared following the protocol described in example 3, test 4, wherein a treatment with the enzyme Mannaway® was added between the steps of liquefaction and of filtration on a bed of diatoms. Indeed, the enzymatic preparation proved to be effective in dissociating high molecular weight PGNs and aggregates.

However, the Mannaway® is contaminated with LPS. In order to eliminate this exogenous contribution, the SD2 resin was replaced by the MN-100 resin, for its efficiency in retaining the LPS-type molecules in the individual tests.

Preparation: waxy starch (containing approximately 30% dry matter) dissolved in process water, left overnight at 4° C., then treated with 0.03% peracetic acid. Removing excess water then taking up in demineralized water adjusted to pH 5.5 (WRAD). Addition of the α-amylase and liquefaction (DEWRAD). Adjustment to pH 8 and treatment with Mannaway® (0.4%) for 24 h at 50° C. (DEWRADM).

The raw material corresponding to the DEWRADM sample was then decontaminated using the following combination:

1. treatment with C extra USP carbon (0.5%) followed by filtration on a sintered glass filter (3 μm),

2. treatment with ENO-PC carbon (0.5%) followed by filtration on a sintered glass filter (3 μm),

3. passage over an MN-100 resin column,

4. sterile filter filtration (0.22 μm).

Samples were taken after the various steps for preparing the raw material, then of the decontamination procedure.

The results of the cell tests are given in FIG. 8.

Before liquefaction, the TLR2 response obtained with the WRAD sample is not saturated, indicating that the treatment with peracetic acid has been effective in reducing the PGN contamination contributed by the process water. On the other hand, liquefaction releases new PGNs, since the TLR2 response is saturated with the DEWRAD sample. LPS contamination is high in the process water (WRAD), and as expected, liquefaction does not significantly alter the TLR4 response. On the other hand, the addition of Mannaway® contributes a significant contamination of exogenous LPS, since the TLR4 response induced by the DEWRADM sample is saturated. The raw material used for this new test is therefore very heavily contaminated with PGN and LPS (FIG. 8A).

The DEWRADM sample was then treated according to the procedure combining the two carbons C-Extra and ENO-PC, the MN-100 resin and the 0.22 μm filter filtration. The initial loads of contaminants contained in the DEWRADM sample were reduced to 100% and the relative reduction percentages were calculated from residual loads after each step (FIG. 8B and table II).

The decontamination procedure was very effective in eliminating LPSs, as a marked reduction in the TLR4 response (>99.8%) was observed, which reaches the threshold limit of detection of the assay. The MN-100 resin therefore retained these contaminants, whether they are contributed by the process water or by the Mannaway® enzymatic preparation.

The combination is also effective in eliminating PGN depolymerization products, given that the threshold limit of detection of the NOD2 response is also reached at the end of the procedure.

On the other hand, a TLR2 response remains (equivalent to 5.2 ng of PGN per g of dry matter), despite a reduction of approximately 99% of the inflammatory load at the end of the method. This data indicates that TLR2 agonists are still present in trace amounts in the raw material, despite the combined effectiveness of the two carbons in removing PGNs.

The response of the Raw cells confirms the presence of inflammatory contaminants in the raw material at the end of the procedure. Indeed, the reduction in the overall inflammatory load is only 98.5%, and the response is significantly above the threshold limit of detection (equivalent to 8 ng of PGN per g of dry matter).

TABLE II
Reduction values (as % of the initial load) and residual
load at the end of the decontamination method

| | HEK-TLR2 | HEK-TLR4 | HEK-NOD2 | Raw |
|---|---|---|---|---|
| Reduction (%) | 98.9 | >99.8 | >90 | 98.4 |
| Residual loads | 5.2 ng/g | <0.5 ng/g | <0.2 ng/g | 8 ng/g |
| | (PGN) | | | (PGN) |

Since previous tests showed the effectiveness of both carbons in eliminating PGNs, even in heavily contaminated raw materials, these results suggest that the Mannaway® enzymatic preparation contributed TLR2 agonists of different chemical nature to the PGNs. These contaminants may be, for example, lipopeptides, known to be strong inducers of TLR2 responses. Thus, the combination of carbons+MN-100 resin, although effective in retaining PGNs and LPSs, would have allowed this type of contaminant through, which would explain the residual TLR2 and Raw responses.

To overcome this problem, the MN-100 resin was replaced by the SD2 resin in the following test, because of its broader spectrum of action.

Procedure 4:

In this test, the raw material corresponds to the DEWRADM sample used for procedure 3. The decontamination procedure uses the previous steps, but replacing the MN-100 resin with the broad-spectrum SD2 resin:

1. treatment with C extra USP carbon (0.5%) followed by filtration on a sintered glass filter (3 μm),

2. treatment with ENO-PC carbon (0.5%) followed by filtration on a sintered glass filter (3 μm),

3. passage on an SD2 resin column,

4. sterile filter filtration (0.22 μm).

Samples were taken after the various steps for preparing the raw material, then of the decontamination procedure.

The results of the cell tests are given in FIG. 9.

Relative reductions for each contaminant were calculated from the residual loads measured after each step of the combination and are expressed as percentages relative to the initial loads contained in the DEWRADM sample (FIG. 9 and table III).

Unlike the combination tested in procedure 3, this combination using SD2 resin makes it possible to extinguish the HEK-TLR2 cell response at the threshold limit of detection, with a reduction in the inflammatory load >99.9%. Thus, the combination of C-extra-USP and ENO-PC carbons in series and SD2 resin apparently has a complementary effect on the elimination of TLR2 agonists, whether of PGN or other type. The procedure also remains effective in removing the NOD agonist contaminants.

The choice of the MN-100 resin was based on the fact that the Mannaway® enzymatic preparation is contaminated with LPSs. Replacement with SD2 resin enables just as effective elimination of the LPSs, with a reduction of >99.9% at the end of the procedure (threshold limit of detection). It can therefore be concluded from these results that SD2 resin is ultimately a better option than MN-100 resin in eliminating LPSs and other contaminants contributed by Mannaway® in the raw material.

Finally, the response of the Raw cells confirms the effectiveness of this procedure in eliminating all the types of contaminants present in the raw material. Indeed, no significant inflammatory response (<threshold limit of detection) is observed any more, which reflects a >99.9% reduction in the overall inflammatory load.

TABLE III
Reduction values (as % of the initial load) and residual
load at the end of the decontamination method

| | HEK-TLR2 | HEK-TLR4 | HEK-NOD2 | Raw |
|---|---|---|---|---|
| Reduction (%) | >99.9 | >99.9 | >90 | >99.9 |
| Residual loads | <1 ng/g | <0.5 ng/g | <0.2 ng/g | <LOD |

LOD, limit of detection

Assessment:

Taken together, the results obtained in this study show that the combination of several production and decontamination steps carefully selected and ordered proves effective in eliminating the inflammatory molecules that may be present in the raw materials used for the preparation of glucose polymers.

The combination comprises the following steps:
suspending the waxy starch at a concentration of between 20 and 40% dry matter in a process water at a pH=5.5,
treating the suspension of starch with a solution of peracetic acid at a final concentration of between 100 and 500 ppm, preferably of 300 ppm,
removing excess water from the starch then taking up in a demineralized water adjusted to pH 5.5 at a concentration of between 20 and 40% of dry matter,
raising the temperature to 107° C. then adding α-amylase for 15 min,
optionally treating with an enzymatic preparation with detergent and clarifying properties, for example Mannaway®, for raw materials heavily loaded with PGN,
filtering the suspension over a bed of diatoms,
treating with an activated carbon with a porosity equivalent to C Extra USP,
treating with a second activated carbon with a porosity equivalent to ENO-PC,
optionally passing over a Dowex-SD2 type adsorption resin for raw materials heavily loaded with LPS and/or treated with an enzymatic preparation with detergent and clarifying properties, for example Mannaway®,
optionally continuous 5 kDa ultrafiltration, for raw materials heavily loaded with PGN depolymerization products,
safety filtration over a sterile filter with a porosity of 0.22 μm.

The combination of these steps makes it possible to target the different families of contaminants and to propose raw materials for glucose polymers which are free of inflammatory reactivity.

### FIGURES

FIG. 1: Cell responses induced by raw materials prepared either in demineralized water or in process water. The results are expressed as absorbance values measured at 620 nm (SEAP test).

FIG. 2: Cell responses induced by unfiltered raw materials and by filtrates obtained after 30 kDa ultrafiltration. The results are expressed as absorbance values measured at 620 nm (SEAP test).

FIG. 3: Cell responses induced by raw materials prepared in demineralized water and treated or not with peracetic acid. The results are expressed as absorbance values measured at 620 nm (SEAP test).

FIG. 4: Cell responses induced by raw materials prepared in process water and treated or not with peracetic acid. The results are expressed as absorbance values measured at 620 nm (SEAP test).

FIG. 5: Cell responses induced by raw materials prepared in process water and treated with Mannaway®. The results are expressed as absorbance values measured at 620 nm (SEAP test).

FIG. 6: (A) Cell responses induced by the raw material prepared for the procedure 1 for decontamination. The results are expressed as absorbance values measured at 620 nm (SEAP test). (B) Reductions in contaminant load during the procedure 1. The load values are obtained from the dose-response curves for each cell type and expressed as percentages relative to those obtained for the DEWRAD sample, reduced to 100%.

FIG. 7: (A) Cell responses induced by the raw material prepared for the procedure 2. The results are expressed as absorbance values measured at 620 nm (SEAP test). (B) Reductions in contaminant load during the “simplified” decontamination procedure. The load values are obtained from the dose-response curves and expressed as percentages relative to those obtained for the DEWRAD sample, reduced to 100%.

FIG. 8: (A) Cell responses induced by the raw material prepared for the procedure 3. The results are expressed as absorbance values measured at 620 nm (SEAP test). (B) Reductions in contaminant load during the procedure 3. The load values are obtained from the dose-response curves and expressed as percentages relative to those obtained for the DEWRADM sample, reduced to 100%.

FIG. 9: Reductions in contaminant load during the procedure 4. The load values are obtained from the dose-response curves and expressed as percentages relative to those obtained for the DEWRADM sample, reduced to 100%.

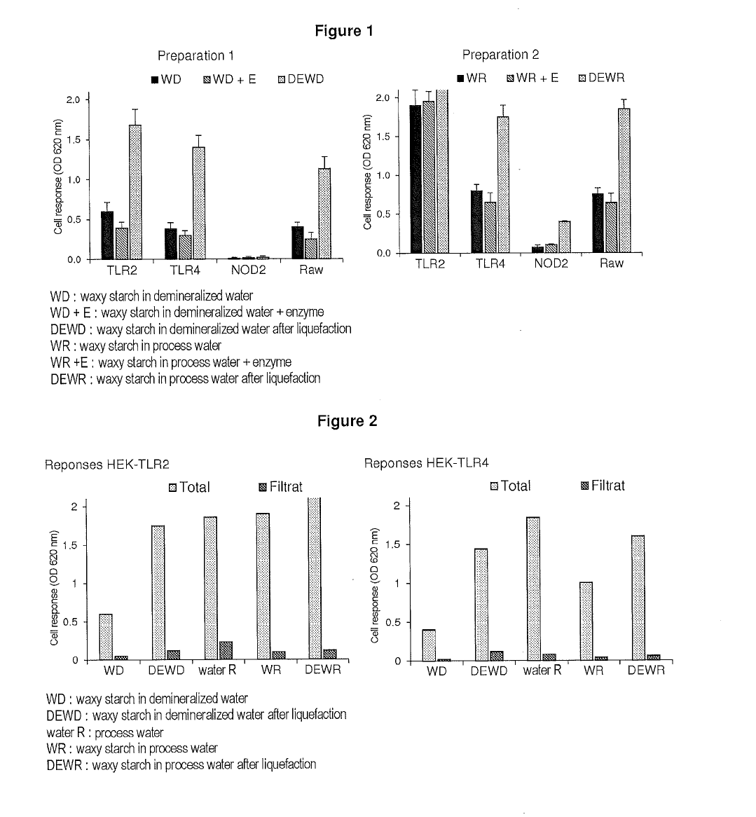

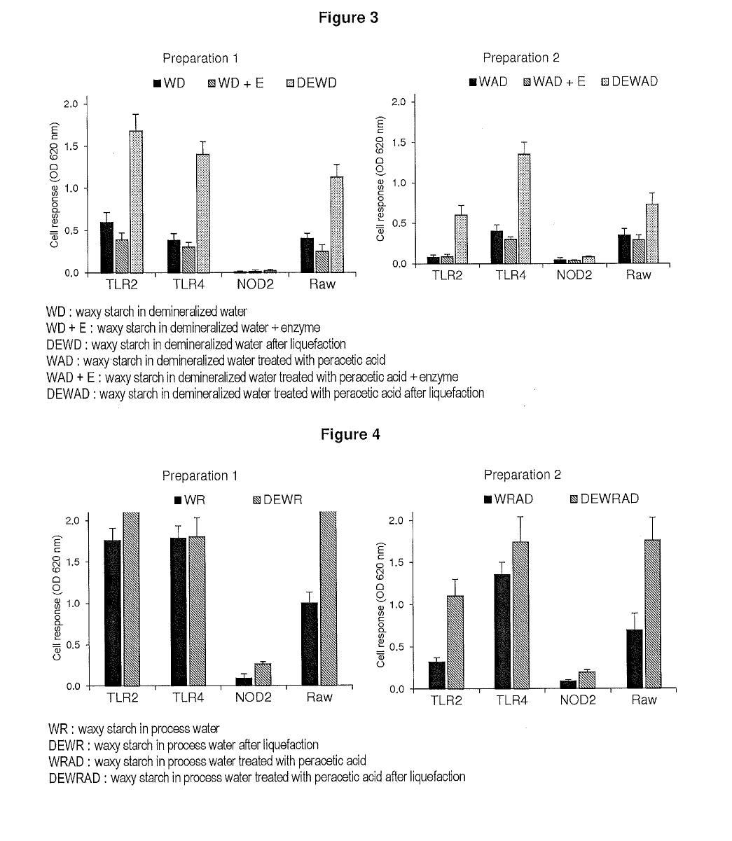

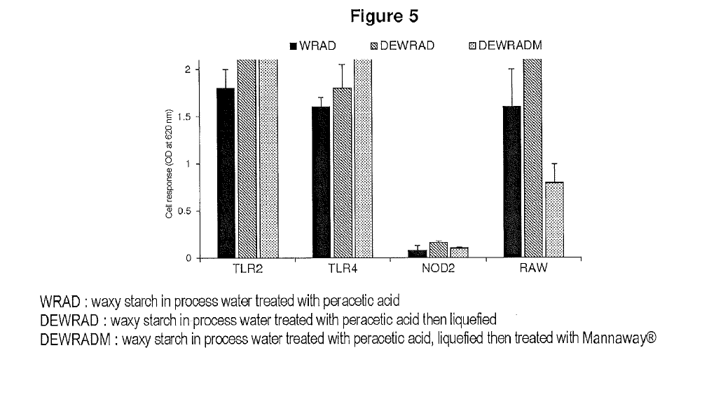

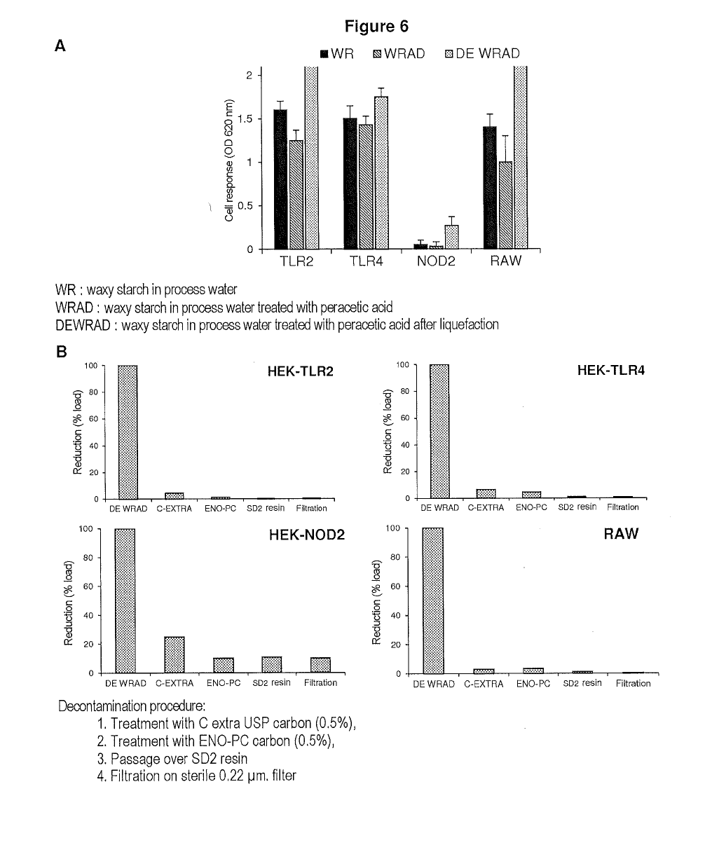

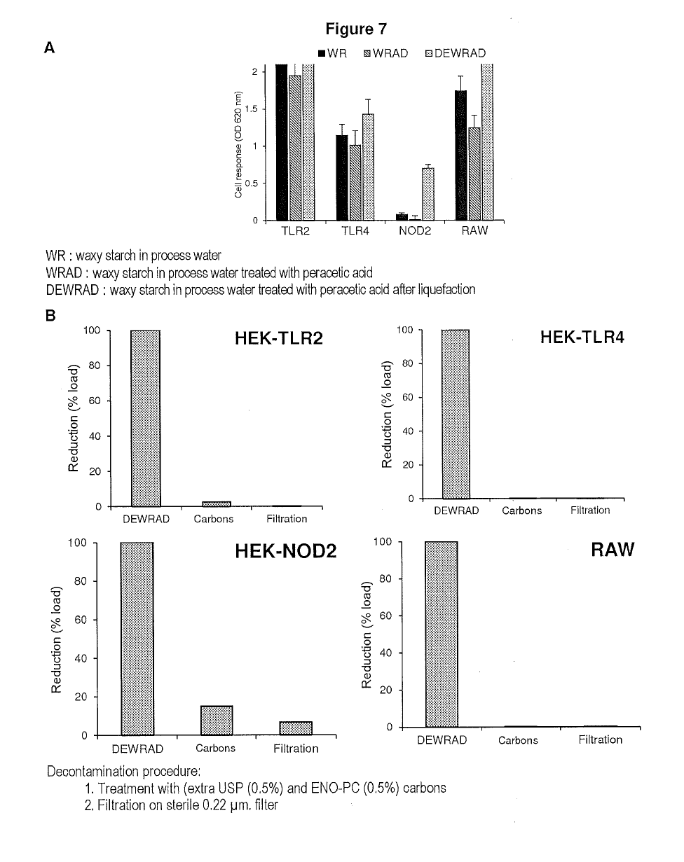

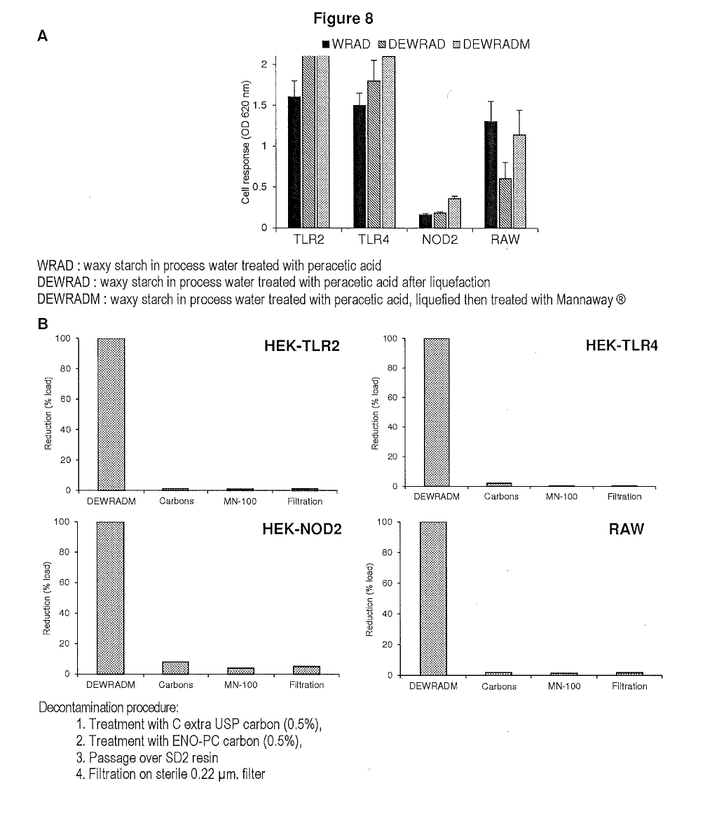

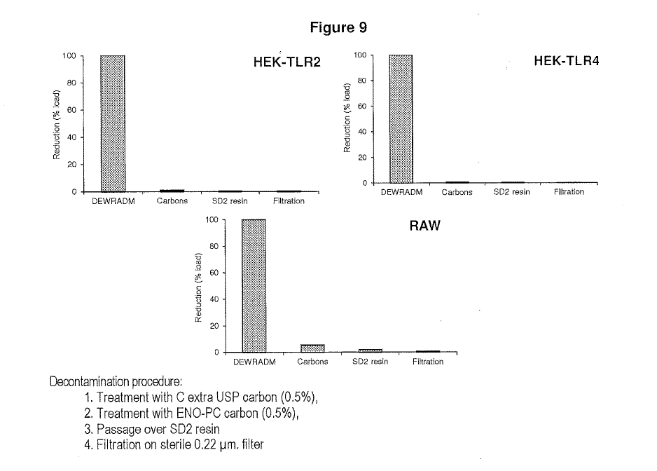

1. A method for decontaminating starches used as raw material for the preparation of glucose polymers intended for peritoneal dialysis, the method comprising:
preparing a waxy starch,
suspending the waxy starch at a concentration of between 20 and 40% dry matter in a process water at a pH of between approximately 5 and approximately 6,
treating the suspension of starch with a solution of peracetic acid at a concentration of between 100 and 500 ppm,
removing excess water from the acid treated starch suspension to form a concentrated suspension and re-suspending the concentrated suspension in a demineralized water adjusted to a pH of between approximately 5 and approximately 6 and to a concentration of between 20 and 40% of dry matter, to provide a liquefied starch,
raising the temperature of the liquefied starch to between approximately 100° C. and 110° C., and adding an α-amylase for approximately 10 to 20 minutes to produces liquefaction products,
optionally further treating the liquefaction products with an enzymatic preparation having detergent and clarifying properties to produce treated liquefaction products,
filtering one of the liquefaction products and treated liquefaction products with a bed of diatoms,
successively treating one of the liquefaction products and treated liquefaction products with a a first and second activated carbon, wherein said first activated carbon is pharmaceutical-grade activated carbon with very high adsorption capacity and “microporous” porosity and said second activated carbon has “mesoporous” porosity to produce activated-carbon treated products,
optionally passing the activated-carbon treated products over a macroporous adsorbent polymer resin having a porosity of greater than 100 angstrom to provide resin treated products, and
optionally, continuously subjecting the resin treated products to 5 kDa ultrafiltration, and
filtering one of said activated-carbon treated products, resin treated products and ultrafiltered products with a sterile filter with a porosity of 0.22 μm.

2. The method according to claim 1, wherein that the enzymatic preparation has enzymatic activity of mannanase type.

3. The method according to claim 1, wherein the treating with an enzymatic preparation is carried out if the liquefied and hydrolyzed starch has a high level of PGN-type contaminants.

4. The method according to claim 1, wherein each optional step is performed.

5. The method according to claim 1, wherein the starch comprises waxy corn starch.

6. A method for decontaminating raw starch materials to prepare glucose polymers for peritoneal dialysis, comprising:
forming a suspension of a waxy starch in one of a demineralized and process water, at a concentration of between 20 and 40% dry matter in the water at a pH of about 5 to about approximately 6,
treating the suspension with a peracetic acid solution at a concentration of between 100 and 500 ppm,
removing excess water from the treated suspension to form a concentrated feed, re-suspending the feed in demineralized water and adjusting to a pH of between approximately 5 and approximately 6 at a starch concentration of between 20 and 40% of dry matter, to provide a second suspension,
raising the temperature of the second suspension to between approximately 100° C. and 110° C., and adding an α-amylase for approximately 10 to 20 minutes to produce liquefaction products,
dissociating high molecular weight aggregates and PGNs in the liquefaction products, followed by filtering, said filtering including at least one of filtering with a bed of diatoms, successive treatments with activated-carbon having pharmaceutical-grade activated carbon with very high adsorption capacity and “macroporous” porosity and mesoporous activated carbon, and 5 kDa resin ultrafiltration, and
sterile filtering with a porosity of 0.22 μm.

7. The method according to claim 1, wherein the water is process water at a pH 5.5, and then treated with 300 ppm peracetic acid.

8. The method according to claim 6, wherein the water is process water at a pH 5.5, and then treated with 300 ppm peracetic acid.

9. The method according to claim 1, wherein said an enzymatic preparation dissociating macrocomplexes, comprising bacterial debris and high molecular weight PGNs.
